SEQUENCE LISTING (20 sequences)

SEQ ID NO: 1 - protein, 21 aa, synthetic construct
MALPVTALLLPLALLLHAARP

SEQ ID NO: 2 - protein, 129 aa, synthetic construct
QVQLVESGGGSVHPGGSLRLSCAGSGFTLAGYAIGWFRQAPGKEREGVSCINSSGGSTYYADSVKGRFTISRDNAKNTVYLQMNSLKPEDTAVYYCAADVYYSGSYLYRSCNPTESKYWGQGTQVTVSS

SEQ ID NO: 3 - protein, 129 aa, synthetic construct
EVQVVESGGGLVQSGGSLRLSCVASGFNLDHYAIGWFRQIPGKEREGVSCISSGGDSTFYIDSVKGRFTISRDNAKNTVYLQMNSLKPEDTAVYHCAADVYYSGNYLYRSCNPHESKYWGQGTQVTVSS

SEQ ID NO: 4 - protein, 129 aa, synthetic construct
EVQVVESGGGLVQPGGSLRLSCAASGFRLNYYAIGWFRQAPGKEREGVSCISSGGDRIYYADSVKGRFTISRDNAKNTVYLQMNSLKPEDTAVYYCAADLYYSGSYYYRSCNPAESGYWGQGTQVTVSS

SEQ ID NO: 5 - protein, 45 aa, synthetic construct
TTTPAPRPPTPAPTIASQPLSLRPEACRPAAGGAVHTRGLDFACD

SEQ ID NO: 6 - protein, 27 aa, synthetic construct
FWVLVVVGGVLACYSLLVTVAFIIFWV

SEQ ID NO: 7 - protein, 41 aa, synthetic construct
RSKRSRLLHSDYMNMTPRRPGPTRKHYQPYAPPRDFAAYRS

SEQ ID NO: 8 - protein, 42 aa, synthetic construct
KRGRKKLLYIFKQPFMRPVQTTQEEDGCSCRFPEEEEGGCEL

SEQ ID NO: 9 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1251 bp, synthetic construct
  source 1..1251: mol_type = other DNA
atggccctccctgtcaccgccctgctgcttccgctggctcttctgctccacgccgctcggcccgaggtgcaggttgtggaatctggcggaggactggttcagtctggcggctctctgagactgagctgtgtggccagcggcttcaacctggatcactatgccatcggctggttcagacagatccccggcaaagagagagagggcgtcagctgtatcagcagcggcggagatagcaccttctacatcgacagcgtgaagggcagattcaccatcagccgggacaacgccaagaacaccgtgtacctgcagatgaacagcctgaagcctgaggacaccgccgtgtatcattgtgccgccgacgtgtactacagcggcaactacctgtacagaagctgcaaccctcacgagagcaagtactggggccagggcacacaagtgaccgtgtcatctaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaagcgcggtcggaagaagctgctgtacatctttaagcaacccttcatgaggcctgtgcagactactcaagaggaggacggctgttcatgccggttcccagaggaggaggaaggcggctgcgaactgcgcgtgaagttcagcagatcagccgatgctcctgcctaccagcagggccagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 11 - protein, 322 aa, synthetic construct
MGWSCIILFLVATATGVHSSLRGRDAPAPTPCVPAECFDLLVRHCVACGLLRTPRPKPAGASSPAPRTALQPQESVGAGAGEAALPLPGLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 969 bp, synthetic construct
  source 1..969: mol_type = other DNA
atgggctggtcctgcatcatcctgtttctggtcgctaccgccacaggcgtccactcctccctgaggggcagagacgcccccgctccaacaccttgcgtgcctgccgagtgcttcgatctgctggtgaggcactgcgtggcctgcggcctgctgaggacccctaggcctaagcccgccggcgctagcagcccagctcctagaacagccctgcagccccaggagagcgtgggcgctggagctggagaggccgctctgcccctgcctggcctggaaccaaaatcttgtgacaaaacccacacatgcccaccttgtcccgcccctgaactgctgggcggaccttctgtctttctgttcccccccaaacccaaggatacactgatgatctctagaacccccgaggtcacatgtgtcgtcgtggatgtgtcccatgaggaccctgaagtgaaattcaactggtacgtggacggagtggaagtccataacgccaaaaccaaaccacgagaggaacagtacaatagcacatatcgagtcgtgtctgtgctgactgtgctgcatcaggattggctgaacggcaaggaatacaaatgtaaagtgtccaataaggcactgcccgctcctattgaaaaaacaatttcaaaggcaaaaggccagcctcgtgaacctcaggtgtacacactgccaccttctcgggaggaaatgaccaaaaaccaggtgtcactgacctgtctggtcaagggcttttacccttccgatattgctgtcgagtgggagagtaacggccagcccgaaaacaactacaaaaccacccctcctgtgctggattccgatggctcattcttcctgtactctaaactgaccgtggataagagtcgctggcagcagggcaatgtgttttcttgctccgtgatgcatgaggcactgcacaaccactacacccagaaatccctgtcactgtctcccggaaaataa

SEQ ID NO: 13 - DNA, 18 bp, synthetic construct
  source 1..18: mol_type = other DNA
cttggtggtcctggctgc

SEQ ID NO: 14 - DNA, 23 bp, synthetic construct
  source 1..23: mol_type = other DNA
ggtacgtgctgttgaactgttcc

SEQ ID NO: 15 - DNA, 47 bp, synthetic construct
  source 1..47: mol_type = other DNA
catgccatgactgtggcccaggcggcccagktgcagctcgtggagtc

SEQ ID NO: 16 - DNA, 47 bp, synthetic construct; identical to SEQ ID NO: 15
  source 1..47: mol_type = other DNA

SEQ ID NO: 17 - DNA, 51 bp, synthetic construct
  source 1..51: mol_type = other DNA
catgccatgactcgcggccggcctggccgctggggtcttcgctgtggtgcg

SEQ ID NO: 18 - DNA, 387 bp, synthetic construct
  source 1..387: mol_type = other DNA
caggtgcagctggttgaatctggcggaggatctgtgcaccctggcggatctctgagactgtcttgtgccggcagcggctttacactggccggatatgccatcggctggttcagacaggcccctggcaaagagagagagggcgtcagctgcatcaatagctctggcggcagcacctactacgccgactctgtgaagggcagattcaccatcagccgggacaacgccaagaacaccgtgtacctgcagatgaacagcctgaagcctgaggacaccgccgtgtactattgtgccgccgacgtgtactacagcggcagctacctgtacagaagctgcaaccccaccgagagcaagtattggggccagggcacacaagtgaccgtgtctagt

SEQ ID NO: 19 - DNA, 387 bp, synthetic construct
  source 1..387: mol_type = other DNA
gaggtgcaggttgtggaatctggcggaggactggttcagtctggcggctctctgagactgagctgtgtggccagcggcttcaacctggatcactatgccatcggctggttcagacagatccccggcaaagagagagagggcgtcagctgtatcagcagcggcggagatagcaccttctacatcgacagcgtgaagggcagattcaccatcagccgggacaacgccaagaacaccgtgtacctgcagatgaacagcctgaagcctgaggacaccgccgtgtatcattgtgccgccgacgtgtactacagcggcaactacctgtacagaagctgcaaccctcacgagagcaagtactggggccagggcacacaagtgaccgtgtcatct

SEQ ID NO: 20 - DNA, 387 bp, synthetic construct
  source 1..387: mol_type = other DNA
gaggtgcaggttgtggaatctggcggaggactggttcagcctggcggatctctgagactgtcttgtgccgccagcggcttccggctgaattactatgccatcggctggttcagacaggcccctggcaaagagagagagggcgtcagctgtatcagctctggcggcgacagaatctactacgccgacagcgtgaagggcagattcaccatcagccgggacaacgccaagaacaccgtgtacctgcagatgaacagcctgaagcctgaggacaccgccgtgtattactgtgccgccgacctgtactacagcggcagctactactacagaagctgcaaccctgccgagagcggctattggggacagggaacacaagtgaccgtgtcctct

(19) United States
(12) Patent Application Publication
(10) Pub. No.: US 2026/0207669 A1
(43) Pub. Date: Jul. 23, 2026

(54) CHIMERIC ANTIGEN RECEPTOR TARGETING BAFFR, CAR-T CELL, AND USE THEREOF

(71) Applicant: Tianjin Medical University General Hospital, Tianjin (CN)

(72) Inventors: Qiang LIU, Tianjin (CN); Guoqiang Chang, Tianjin (CN)

(21) Appl. No.: 19/454,751

(22) Filed: Jan. 21, 2026

Related U.S. Application Data
(63) Continuation of application No. PCT/CN2024/089326, filed on Apr. 23, 2024.

(30) Foreign Application Priority Data
Oct. 13, 2023 (CN) 202311321640.8

(51) Int. Cl. A61K 35/17; A61K 40/11; A61K 40/31; A61K 40/42; C07K 14/705; C07K 16/28; C12N 5/0783; C12N 15/86

(52) U.S. Cl. CPC A61K 35/17; A61K 40/11; A61K 40/31; A61K 40/4215; C07K 14/7051; C07K 14/70517; C07K 14/70521; C07K 14/70578; C07K 16/2878; C12N 5/0636; C12N 15/86; A61K 2239/13; A61K 2239/17; A61K 2239/21; C07K 2317/53; C07K 2317/569; C07K 2319/02; C07K 2319/03; C12N 2740/15043

(57) ABSTRACT

The present disclosure discloses a chimeric antigen receptor targeting BAFFR, a CAR-T cell, and use thereof. The chimeric antigen receptor targeting BAFFR includes, from the N end to the C end, a signal peptide region, an antigen binding domain targeting BAFFR, a hinge region, a transmembrane domain, a costimulatory domain and a signal transduction domain connected in sequence. The costimulatory domain includes a CD28 costimulatory domain and a 4-1BB costimulatory domain which are connected, and the signal transduction domain is ITAM1 of CD3ζ. According to the present disclosure, CAR-T cells are constructed based on BAFFR. By using the mechanism of antigen-antibody binding, autoimmune B cells or malignantly proliferated B cells in patients with autoimmune diseases or some tumors are effectively depleted, thus providing a new option for the treatment of human autoimmune diseases and some hematological tumors.

### CROSS-REFERENCE TO RELATED APPLICATIONS

This application is a continuation of International Application No. PCT/CN2024/089326, filed on Apr. 23, 2024, which claims priority to Chinese Patent Application No. 202311321640.8, filed on Oct. 13, 2023. The disclosures of the aforementioned applications are hereby incorporated by reference in their entireties.

### STATEMENT OF SEQUENCE DISCLOSURE

The Sequence Listing XML, which is part of this application, is incorporated herein by reference in its entirety. The Sequence Listing XML is submitted as a file named PR34293WHUS_Sequence_listing.xml, created on Oct. 21, 2025, and having a size of 22528 bytes.

### TECHNICAL FIELD

The present disclosure relates to the technical field of molecular biology and medical science, and specifically to a chimeric antigen receptor targeting BAFFR, a CAR-T cell, and use thereof.

### BACKGROUND

In the chimeric antigen receptor (CAR)-T cell (CAR-T) therapy, a chimeric antigen receptor expression vector is constructed by genetic engineering and transfected into T cells, so that a chimeric antibody recognizing a target cell can be expressed on the surface of T cells, and a specific killing effect on the target cell can be achieved by specifically recognizing an antigen on the surface of the target cell.

Compared with other immune cell therapies, CAR-T cell technology has more advantages. Firstly, CAR-T cells get closer to the target cells and attack the target cells directly and accurately. Secondly, CAR-T cells can kill target cells independently of MHC and require no antigen presentation mechanism to identify them, so that the immune escape mediated by the down-regulation of MHC and the reduction of antigen presentation is overcome, and the target cells can be killed more effectively. Thirdly, the construction of a CAR gene is based on the target antigen expressed by the target cell. CAR-T can utilize both a protein antigen and a glycolipid non-protein antigen, thus expanding the target range of the target antigen. Finally, therapeutic effect is long lasting. A gene sequence that promotes the proliferation and activation of T cells can be added to the CAR-T structure, to ensure the proliferation of T cells after entering the human body. CAR-T cells have immune memory function and can survive in the body for a long time.

In recent years, CAR-T therapy is mostly used for tumor treatment. It has many incomparable advantages over other therapies and is considered as one of the most promising therapies for cancers. However, CAR-T immunotherapy is greatly limited for the treatment of other diseases such as autoimmune diseases, mainly due to the discovery of new targets. Studies have shown that BAFF is involved in the progress of many autoimmune diseases, and has a significantly increased expression level in many autoimmune diseases. For example, BAFF is constantly highly expressed in patients with systemic lupus erythematosus, and the concentration of BAFF in the patients' serum is positively correlated with the titer of anti-dsDNA antibodies. In addition, the expression level of BAFF in the sera of patients with Sjogren's syndrome, rheumatoid arthritis and multiple sclerosis is also increased significantly. BAFF can bind to three membrane receptors of the TNFR family on the surface of B cells, namely, BAFFR, TACI and BCMA. However, the binding of BAFF and BAFFR plays the most important role in the maturation of peripheral B cells. Therefore, the construction of CAR-T cells targeting BAFFR will be an important breakthrough in the treatment of autoimmune diseases and/or hematological tumors.

### SUMMARY

In view of the defects or disadvantages in the related art, it is desirable to provide a chimeric antigen receptor targeting BAFFR, a CAR-T cell, and use thereof. Through the phage display technology in combination with a high-throughput expression platform, a nanobody targeting BAFFR is screened out, which has the advantages of high affinity, high stability, low molecular weight, low immunogenicity and powerful permeability. According to the obtained BAFFR antibody sequence, a CAR-T cell is constructed, which can specifically target autoimmune B cells and avoid immune deficiency and infection of patients caused by B cell depletion. Moreover, the B cell depletion may reduce the patients' response to vaccines, such as COVID-19 vaccine. In contrast, CAR-T cells only target autoreactive B cells, which can effectively avoid the low response to vaccines caused by B cell depletion and provide a new option for the treatment of human autoimmune-related diseases.

The present disclosure provides a chimeric antigen receptor targeting BAFFR, which includes, from the N end to the C end, a signal peptide region, an antigen binding domain targeting BAFFR, a hinge region, a transmembrane domain, a costimulatory domain and a signal transduction domain connected in sequence. The costimulatory domain includes a CD28 costimulatory domain and a 4-1BB costimulatory domain which are connected, and the signal transduction domain is ITAM1 of CD3ζ.

Further, the signal peptide region has an amino acid sequence as shown in SEQ ID No: 1.

Further, the antigen binding domain targeting BAFFR is a nanobody targeting BAFFR, and the nanobody targeting BAFFR has an amino acid sequence as shown in SEQ ID No: 2, SEQ ID No: 3, or SEQ ID No: 4.

Further, the hinge region is a CD8 hinge domain having an amino acid sequence as shown in SEQ ID No: 5; and/or
the transmembrane domain is a CD28 transmembrane domain having an amino acid sequence as shown in SEQ ID No: 6; and/or
the CD28 costimulatory domain has an amino acid sequence as shown in SEQ ID No: 7; and/or
the 4-1BB costimulatory domain has an amino acid sequence as shown in SEQ ID No: 8; and/or
the ITAM1 of CD3ζ has an amino acid sequence as shown in SEQ ID No: 9.

In addition, the present disclosure further provides an isolated nucleic acid. The isolated nucleic acid includes a nucleotide sequence for expressing the chimeric antigen receptor targeting BAFFR.

Further, the isolated nucleic acid includes a nucleic acid fragment having a nucleotide sequence as shown in SEQ ID NO: 10.

In addition, the present disclosure further provides a recombinant vector including the isolated nucleic acid.

In addition, the present disclosure further provides a CAR-T cell. The CAR-T cell includes the isolated nucleic acid, or the CAR-T cell is a cell transformed with the recombinant vector.

In addition, the present disclosure further provides use of the chimeric antigen receptor targeting BAFFR in drugs, including use of the chimeric antigen receptor targeting BAFFR, the isolated nucleic acid, the recombinant vector or the CAR-T cell in preparing drugs for treating immune-related diseases.

In addition, the present disclosure further provides a pharmaceutical composition, which includes an expression vector for expressing the chimeric antigen receptor targeting BAFFR or the CAR-T cell.

Compared with the prior art, the present disclosure has the following beneficial effects:
(1) At present, B cell depletion by CD19 or CD20 antibody is the main treatment for autoimmune diseases, but the treatment effect is undesirable. In one aspect, the CD19 or CD20 antibody will target all naive and mature B cells, which will easily lead to immune deficiency and even infection in patients. In another aspect, the CD19 or CD20 antibody may reduce the patients' response to vaccines, such as COVID-19 vaccine. According to the present disclosure, CAR-T cells are constructed based on BAFFR, which can target auto-reactive B cells, especially plasma cells, rather than depleting all B cells. As a result, the basic autoimmune ability and response to vaccines of the patients are reserved.
(2) Autoantibodies exist in patients with autoimmune diseases, such as dsDNA antibody in patients with systemic lupus erythematosus, MOG antibody in patients with multiple sclerosis, and AQP4 antibody in patients with neuromyelitis optica. CD20 monoclonal antibody cannot reduce the concentration of these pathogenic autoantibodies (30-70%) in the patients. According to the present disclosure, CAR-T cells are constructed based on BAFFR, which mainly target plasma cells producing autoantibodies, and can effectively reduce the concentration of autoantibodies in patients.
(3) In the present disclosure, through the phage display technology in combination with a high-throughput expression platform, a nanobody targeting BAFFR is screened out, which has the advantages of high affinity, high stability, low molecular weight, low immunogenicity and powerful permeability.

It is to be understood that the description in the summary is not intended to define key or important features of embodiments of the present disclosure, and not intended to limit the scope of the present disclosure. Other features of the present disclosure will be readily understood from the following description.

### BRIEF DESCRIPTION OF THE DRAWINGS

Other features, objects, and advantages of the present disclosure will become apparent upon reading the detailed description of non-limiting embodiments that follow with reference to the accompanying drawings.

FIG. 1 is a schematic diagram showing the binding activity of anti-BAFFR antibody to BAFFR determined by ELISA;

FIG. 2 is a schematic diagram showing the structure of CAR-T cells, in which BAFFR-CAR represents a chimeric antigen receptor polypeptide targeting BAFFR, which includes, from the N end to the C end, a CD8a signal peptide region, anti-BAFFR VHH, a CD8 hinge region, a CD28 transmembrane region, a CD28 costimulatory domain, a 4-1BB costimulatory domain and a CD3ζ signal transduction domain;

FIG. 3 is a schematic diagram showing the rate of T cells transfected with CAR plasmid detected by flow cytometry;

FIG. 4 compares the killing effects of BAFFR-CAR-T and the control BAFFR-CAR-T on Nalm-6-Luc cells at various effector-to-target ratios in the example; and

FIG. 5 compares the levels of IL-2 and IFN-γ secreted in the presence of BAFFR-CAR-T and the control BAFFR-CAR-T at various effector-to-target ratios in the example.

### DETAILED DESCRIPTION

Hereinafter, the present disclosure is described in further detail with reference to drawings and examples. It can be understood that the specific embodiments described herein are merely used to explain the present disclosure, and do not constitute restriction on the present disclosure. In addition, it should be further noted that for ease of description, only the parts relevant to the present disclosure are shown in the drawings.

It is to be understood that where there are no contradictions, the embodiments and the features in the embodiments of the present disclosure can be combined with each other without conflict. Hereinafter, the present disclosure will be described in detail by way of examples in connection with drawings.

An embodiment of the present disclosure provides a chimeric antigen receptor targeting BAFFR, which includes, from the N end to the C end, a signal peptide region, an antigen binding domain targeting BAFFR, a hinge region, a transmembrane domain, a costimulatory domain and a signal transduction domain connected in sequence. The costimulatory domain includes a CD28 costimulatory domain and a 4-1BB costimulatory domain which are connected, and the signal transduction domain is ITAM1 of CD3ζ.

In some embodiments, the hinge region is a hinge region at least selected from: CD28, CD3ζ, CD45, CD4, CD5, CD8, CD9, CD16, CD22, CD33, CD37, CD134, CD137, ICOS and CD154.

In some embodiments, the transmembrane domain is a transmembrane domain at least selected from: a, B or (chain of T cell receptor, CD28, CD3ζ, CD45, CD4, CD5, CD8, CD9, CD16, CD22, CD33, CD37, CD64, CD80, CD86, CD134, CD137, CD154, KIRDS2, OX40, CD2, CD27, ICOS, GITR, CD40, BAFFR, HVEM, SLAMF7, NKp80, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, LFA-1, ITGAM, CD11b, ITGAX, CD11c, ITGB1, CD29, ITGB2, ITGB7, TNFR2, DNAM1, SLAMF4, CD84, CD96, CEACAM1, CRTAM, Ly9, PSGL1, CD100, SLAMF6, SLAM, BLAME, SELPLG, LTBR, PAG/Cbp, NKp44, NKp30, NKp46, NKG2D and NKG2C.

In a preferred embodiment, the signal peptide region has an amino acid sequence as shown in SEQ ID No: 1.

SEQ ID No: 1 (the amino acid sequence of the signal peptide region) is:
MALPVTALLLPLALLLHAARP.

In a preferred embodiment, the antigen binding domain targeting BAFFR is a nanobody targeting BAFFR, and the nanobody targeting BAFFR has an amino acid sequence as shown in SEQ ID No: 2, SEQ ID No: 3, or SEQ ID No: 4.

SEQ ID No: 2 (the amino acid sequence of NB467-7) is:
QVQLVESGGGSVHPGGSLRLSCAGSGFTLAGYAIGWFRQAPGKEREGVSCINSSG
GSTYYADSVKGRFTISRDNAKNTVYLQMNSLKPEDTAVYYCAADVYYSGSYLYRSC
NPTESKYWGQGTQVTVSS;
SEQ ID No: 3 (the amino acid sequence of NB467-71) is:
EVQVVESGGGLVQSGGSLRLSCVASGFNLDHYAIGWFRQIPGKEREGVSCISSGG
DSTFYIDSVKGRFTISRDNAKNTVYLQMNSLKPEDTAVYHCAADVYYSGNYLYRSCN
PHESKYWGQGTQVTVSS;
SEQ ID No: 4 (the amino acid sequence of NB467-88) is:
EVQVVESGGGLVQPGGSLRLSCAASGFRLNYYAIGWFRQAPGKEREGVSCISSG
GDRIYYADSVKGRFTISRDNAKNTVYLQMNSLKPEDTAVYYCAADLYYSGSYYYRS
CNPAESGYWGQGTQVTVSS.

In a preferred embodiment, the hinge region is a CD8 hinge domain having an amino acid sequence as shown in SEQ ID No: 5; and/or
the transmembrane domain is a CD28 transmembrane domain having an amino acid sequence as shown in SEQ ID No: 6; and/or
the CD28 costimulatory domain has an amino acid sequence as shown in SEQ ID No: 7; and/or
the 4-1BB costimulatory domain has an amino acid sequence as shown in SEQ ID No: 8; and/or
the ITAM1 of CD3 has an amino acid sequence as shown in SEQ ID No: 9.

SEQ ID No: 5 (the CD8 hinge domain) is:
TTTPAPRPPTPAPTIASQPLSLRPEACRPAAGGAVHTRGLDFACD;
SEQ ID No: 6 (the CD28 transmembrane domain) is
FWVLVVVGGVLACYSLLVTVAFIIFWV;
SEQ ID No: 7 (the amino acid sequence of CD28 costimulatory domain) is:
RSKRSRLLHSDYMNMTPRRPGPTRKHYQPYAPPRDFAAYRS;
SEQ ID No: 8 (the amino acid sequence of 4-1BB costimulatory domain) is:
KRGRKKLLYIFKQPFMRPVQTTQEEDGCSCRFPEEEEGGCEL; and
SEQ ID No: 9 (the amino acid sequence of ITAM1 of CD3ζ) is:
RVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQA
LPPR.

In addition, an embodiment of the present disclosure further provides an isolated nucleic acid. The isolated nucleic acid includes a nucleotide sequence for expressing the chimeric antigen receptor targeting BAFFR.

In a preferred embodiment, the isolated nucleic acid includes a nucleic acid fragment having a nucleotide sequence as shown in SEQ ID NO: 10.

SEQ ID No: 10 is:
ATGGCCCTCCCTGTCACCGCCCTGCTGCTTCCGCTGGCTCTTCTGCTCCACGC
CGCTCGGCCC
GAGGTGCAGGTTGTGGAATCTGGCGGAGGACTGGTTCAGTCTGGCGGCTCTC
TGAGACTGAGCTGTGTGGCCAGCGGCTTCAACCTGGATCACTATGCCATCGGCTG
GTTCAGACAGATCCCCGGCAAAGAGAGAGAGGGCGTCAGCTGTATCAGCAGCGG
CGGAGATAGCACCTTCTACATCGACAGCGTGAAGGGCAGATTCACCATCAGCCGG
GACAACGCCAAGAACACCGTGTACCTGCAGATGAACAGCCTGAAGCCTGAGGAC
ACCGCCGTGTATCATTGTGCCGCCGACGTGTACTACAGCGGCAACTACCTGTACAG
AAGCTGCAACCCTCACGAGAGCAAGTACTGGGGCCAGGGCACACAAGTGACCGT
GTCATCT
ACCACGACGCCAGCGCCGCGACCACCAACACCGGCGCCCACCATCGCGTCGC
AGCCCCTGTCCCTGCGCCCAGAAGCGTGCCGGCCAGCGGCGGGGGGCGCAGTGC
ACACGAGGGGGCTGGACTTCGCCTGTGAT
TTCTGGGTGCTGGTCGTTGTGGGCGGCGTGCTGGCCTGCTACAGCCTGCTGGT
GACAGTGGCCTTCATCATCTTTTGGGTG
AGGAGCAAGCGGAGCAGACTGCTGCACAGCGACTACATGAACATGACCCCCC
GGAGGCCTGGCCCCACCCGGAAGCACTACCAGCCCTACGCCCCTCCCAGGGATTT
CGCCGCCTACCGGAGC
AAGCGCGGTCGGAAGAAGCTGCTGTACATCTTTAAGCAACCCTTCATGAGGC
CTGTGCAGACTACTCAAGAGGAGGACGGCTGTTCATGCCGGTTCCCAGAGGAGG
AGGAAGGCGGCTGCGAACTG
CGCGTGAAGTTCAGCAGATCAGCCGATGCTCCTGCCTACCAGCAGGGCCAGA
ACCAGCTGTACAACGAGCTGAACCTGGGGAGAAGAGAAGAGTACGACGTGCTGG
ACAAGCGGAGAGGCAGAGATCCTGAGATGGGCGGCAAGCCCAGACGGAAGAATC
CTCAAGAGGGCCTGTATAATGAGCTGCAGAAAGACAAGATGGCCGAGGCCTACAG
CGAGATCGGAATGAAGGGCGAGCGCAGAAGAGGCAAGGGACACGATGGACTGTA
CCAGGGCCTGAGCACCGCCACCAAGGATACCTATGATGCCCTGCACATGCAGGCC
CTGCCTCCAAGA.

In addition, an embodiment of the present disclosure further provides a recombinant vector including the isolated nucleic acid.

In addition, an embodiment of the present disclosure further provides a CAR-T cell. The CAR-T cell includes the isolated nucleic acid, or the CAR-T cell is a cell transformed with the recombinant vector.

In addition, an embodiment of the present disclosure further provides use of the chimeric antigen receptor targeting BAFFR in drugs, including use of the chimeric antigen receptor targeting BAFFR, the isolated nucleic acid, the recombinant vector or the CAR-T cell in preparing drugs for treating immune-related diseases.

The immune-related diseases include autoimmune diseases and tumors.

The autoimmune diseases include immune diseases of the nervous system, such as multiple sclerosis, neuromyelitis optica, and myasthenia gravis, and other autoimmune diseases such as rheumatoid arthritis, systemic lupus erythematosus, and Sjogren's syndrome.

The tumors include hematomas, such as B-cell lymphomas, such as Hodgkin's lymphoma and nodular lymphocyte-predominant Hodgkin lymphoma, and non-Hodgkin's lymphomas, such as diffuse large B-cell lymphoma, follicular lymphoma, mucosa-associated lymphoid tissue lymphoma (MALT), small lymphocytic lymphoma/chronic lymphocytic leukemia, and, mantle cell lymphoma (MCL).

In addition, an embodiment of the present disclosure also provides a pharmaceutical composition, which includes an expression vector for expressing the chimeric antigen receptor targeting BAFFR or the CAR-T cell.

### Example 1: Production of Nanobodies Against BAFFR by Phage Display

In this example, a variety of antibody coding fragments were obtained by immunizing alpaca with human BAFFR as an antigen, and then a candidate antibody clone was screened by a phage library and a mammalian expression system. Specifically, specific steps of the screening process of the anti-BAFFR nanobodies were as follows.

a. Preparation of Antigen

According to the amino acid sequence and nucleotide sequence of human BAFFR, the antigen that can effectively induce alpacas to produce specific antibodies against human BAFFR was analyzed and designed. Human IgG1 Fc was linked to the C end, to obtain a modified antigen, which was designated as “human BAFFR hFC antigen”. The amino acid sequence is as shown in SEQ ID No: and 11, and the nucleotide sequence is as shown in SEQ ID No: 12.

SEQ ID No: 11 (the amino acid sequence of human BAFFR hFC antigen) is:
MGWSCIILFLVATATGVHSSLRGRDAPAPTPCVPAECFDLLVRHCVACGLLRTPRP
KPAGASSPAPRTALQPQESVGAGAGEAALPLPGLEPKSCDKTHTCPPCPAPELLGGPS
VFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK;
SEQ ID No: 12 (the nucleotide sequence of human BAFFR hFC antigen) is:
ATGGGCTGGTCCTGCATCATCCTGTTTCTGGTCGCTACCGCCACAGGCGTCCA
CTCCTCCCTGAGGGGCAGAGACGCCCCCGCTCCAACACCTTGCGTGCCTGCCGAG
TGCTTCGATCTGCTGGTGAGGCACTGCGTGGCCTGCGGCCTGCTGAGGACCCCTA
GGCCTAAGCCCGCCGGCGCTAGCAGCCCAGCTCCTAGAACAGCCCTGCAGCCCCA
GGAGAGCGTGGGCGCTGGAGCTGGAGAGGCCGCTCTGCCCCTGCCTGGCCTGGA
ACCAAAATCTTGTGACAAAACCCACACATGCCCACCTTGTCCCGCCCCTGAACTG
CTGGGCGGACCTTCTGTCTTTCTGTTCCCCCCCAAACCCAAGGATACACTGATGAT
CTCTAGAACCCCCGAGGTCACATGTGTCGTCGTGGATGTGTCCCATGAGGACCCT
GAAGTGAAATTCAACTGGTACGTGGACGGAGTGGAAGTCCATAACGCCAAAACC
AAACCACGAGAGGAACAGTACAATAGCACATATCGAGTCGTGTCTGTGCTGACTG
TGCTGCATCAGGATTGGCTGAACGGCAAGGAATACAAATGTAAAGTGTCCAATAA
GGCACTGCCCGCTCCTATTGAAAAAACAATTTCAAAGGCAAAAGGCCAGCCTCGT
GAACCTCAGGTGTACACACTGCCACCTTCTCGGGAGGAAATGACCAAAAACCAG
GTGTCACTGACCTGTCTGGTCAAGGGCTTTTACCCTTCCGATATTGCTGTCGAGTG
GGAGAGTAACGGCCAGCCCGAAAACAACTACAAAACCACCCCTCCTGTGCTGGA
TTCCGATGGCTCATTCTTCCTGTACTCTAAACTGACCGTGGATAAGAGTCGCTGGC
AGCAGGGCAATGTGTTTTCTTGCTCCGTGATGCATGAGGCACTGCACAACCACTA
CACCCAGAAATCCCTGTCACTGTCTCCCGGAAAATAA.

b. Immunization of Alpaca

The human BAFFR hFC antigen obtained in Step a was completely and evenly mixed with an equal volume of Freund's adjuvant, and subcutaneously injected into alpaca.

Specifically, on day 1, alpacas were immunized with an emulsified mixture of 500 μg of human BAFFR hFC antigen with an equal volume of Freund's complete adjuvant, and four times of booster immunization were carried out with an emulsified mixture of 250 μg of human BAFFR hFC antigen with an equal volume of Freund's complete adjuvant on days 21, 42, 63, and 84 respectively. 7 days after the first 4 immunizations, 10 mL of peripheral blood was collected from alpacas and the anti-BAFFR serum titer in the blood was detected by ELISA.

The specific steps of ELISA detection were as follows: Human BAFFR his antigen was diluted to 2 μg/mL with a 0.05 M carbonate buffer (pH=9.6) and added to a plate in an amount of 100 L/well. The plate was coated overnight at 4° C. The coating solution was discarded and the plate was washed 3 times with PBST. 300 μL of 5% skim milk was added to each well and the plate was blocked at 37° C. for 1 h. The plate was washed 3 times with a PBST buffer. 100 μL/well of a serum diluent (fold-dilution from 1:2000) was added and incubated at 37° C. for 45 min. The plate was washed 5 times with PBST. 100 μL goat anti-alpaca IgG (H+L) HRP (Chengdu NBbiolab Biotechnology Co., Ltd., Cat #: S001H, Chengdu NBbiolab Biotechnology Co., Ltd., 1:1 W dilution with PBS) was added to each well, and incubated at 37° C. for 45 min. The plate was washed 5 times with PBST. A TMB developing solution (100 μL/well) was added and incubate at 37° C. for 5 min. The reaction was terminated by adding a termination solution (50 μL/well), and the optical density was measured at 450 nm.

1 week after the 5th immunization, 50 mL of peripheral blood was collected from alpaca and mononuclear cells were isolated.

### C. Library Construction

RNA of PBMCs obtained in Step b was extracted, and a target gene fragment was obtained by nested PCR after reverse transcription. The target gene fragment was cloned into a eukaryotic expression vector, and the obtained expression vector was transformed into competent cells to construct a BAFFR-VHH phage display library. The specific steps were as follows.

PBMC cells screened in Step b were used as a template total RNA was extracted from the cells by using RNAiso Plus, and the RNA was reverse transcribed into cDNA by using PrimeScript™ II 1st Strand cDNA Synthesis Kit. The target fragment was obtained by nested PCR. The amplification system for the target fragment was shown in Table 1 below, and the amplification procedure was shown in Table 2 below.

TABLE 1
First-round reaction system of nested PCR

| Reagent | Volume (μL) |
|---|---|
| cDNA template (5-fold dilution) | 5 |
| Upstream primer: NPR-19001 (SEQ ID No: 13) | 1.5/1.5 |
| 5′ CTTGGTGGTCCTGGCTGC 3′ | |
| Downstream primer: NPR-19002 (SEQ ID No: 14) | |
| 5′ GGTACGTGCTGTTGAACTGTTCC 3′ | |
| dNTP Mixture | 4 |
| 10×PrimeSTAR Buffer (Mg2+ plus) | 5 |
| PrimeSTAR ® HS DNA Polymerase | 0.5 |
| ddH2O | q.s. to 50 |

TABLE 2
Second-round reaction system of nested PCR

| Reagent | Volume (μL) |
|---|---|
| cDNA template (5-fold dilution) | 5 |
| Upstream primer: NPR-19003 (SEQ ID No: 15) | 1.5/1.5 |
| 5′-CATGCCATGACTGTGGCCCAGGCGGCCCAGKTGCAGCTCGTGGAG | |
| TC-3′ | |
| Downstream primer: NPR-19004 (SEQ ID No: 16) | |
| 5′-CATGCCATGACTGTGGCCCAGGCGGCCCAGKTGCAGCTCGTGGAG | |
| TC-3′ or | |
| NPR-19005 (SEQ ID No: 17) | |
| 5′-CATGCCATGACTCGCGGCCGGCCTGGCCGCTGGGGTCTTCGCTGTG | |
| GTGCG-3′ | |
| dNTP Mixture | 4 |
| 10×PrimeSTAR Buffer (Mg2+ plus) | 5 |
| PrimeSTAR ® HS DNA Polymerase | 0.5 |
| ddH2O | q.s. to 50 |

The amplified nucleotide fragment encoding alpaca VHH was cloned into the eukaryotic expression vector pComb3XSS (Chengdu NBbiolab Biotechnology Co., Ltd.). The produced recombinant vector was transformed into TGI competent cells by electric shock, to obtain a VHH phage display library. To further identify whether the BAFFR-VHH phage display library was successfully constructed, the library was cultured on 2-YT-A plate, and 48 clones were picked up from the formed colonies for sequencing.

The sequencing results shows that the successful insertion rate of the clone is 100%. According to the number of library transformants, the library insertion rate and the diversity sequencing results, the library capacity is calculated to be 1.92×10⁹, showing that the library capacity and diversity of the obtained BAFFR-VHH phage display library are good.

d. Nanobody Production

The phage display library obtained in Step c was used for panning and positive clones were screened. The coding sequence of VHH antibody screened was fused with the coding sequence of Human IgG1 Fc, constructed into the expression vector of pTT5 (Chengdu NBbiolab Biotechnology Co., Ltd.) and transfected into eukaryotic cells, followed by expression and purification. Finally, anti-human BAFFR nanobody was obtained. The specific method was as follows.

SA-magnetic beads (Suzhou Nanowin Technology Co., Ltd., MPHTSA-300) were taken, and washed 2 times with PBS. Bio-Human BAFFR-his (Acro, BAR-H82E3) antigen was diluted with PBS to a final concentration of 5 μg/mL, and 2×10¹¹ phage library was added, and incubated at 37° C. for 1 h. The incubated mixture was added to a magnetic bead-conjugated tube and shaken at 4° C. for 45 min. The supernatant was removed under the action of a magnetic frame, and the magnetic beads were washed 3 times with PBST and then 2 times with PBS. 800 μL of Gly-HCl eluent was added, and incubated at 37° C. for 8 min, to elute the specifically bound phage. The eluate was transferred to a 1.5 mL sterile centrifuge tube and quickly neutralized with 160 μL of a Tris-HCl neutralization buffer. 10 μL was diluted over gradients, the titer was determined, and the panning recovery rate was calculated. The remaining eluates were mixed, amplified, and purified, for the next round of affinity panning.

After two rounds of screening, the supernatant of the monoclonal phage was identified by ELISA after the second round of screening. Bio-Human BAFFR-his antigen was immobilized on a 96-well microplate according to a specification of 2 μg/mL*100 μL, and incubated at 4° C. overnight. The coating solution was discarded and the plate was washed 3 times with PBST. 300 μL of 5% skim milk was added to each well and the plate was blocked 37° C. for 1 h. The plate was washed 3 times with a PBST buffer. Two-fold dilutions of the phage supernatant were added in an amount of 100 μL/well, and incubated at 37° C. for 45 min. The plate was washed 5 times with PBST. Mouse anti-M13 antibody HRP (Chengdu NBbiolab Biotechnology Co., Ltd.) was added to each well in an amount of 100 μL/well and incubated at 37° C. for 45 min. The plate was washed 5 times with PBST. ATMB developing solution (100 μL/well) was added and incubated at 37° C. for 5 min.

The reaction was terminated by adding a 1 M HCl termination solution in an amount of 50 μL/well, and the optical density was detected at a wavelength of 450 nm. The positive clones (OD450>1) were screened according to the detection results. Human IgG1 Fc was fused to the positive clone and constructed into pTT5 plasmid. The corresponding plasmid was transfected into mammalian cell HEK293T, and expressed on a shaker with 5% carbon dioxide at 37° C. for 7 days. The cell supernatant was collected after culture, and 3 target antibodies were isolated and purified by Protein A affinity packing (Suzhou Nanowin Technology Co., Ltd., 17010-050100), which were designated as NB467-7, NB467-71, and NB467-88 respectively.

The VHH amino acid sequences and nucleotide sequences of nanobodies NB467-7, NB467-71, and NB467-88 are as follows:

SEQ ID No: 2 (the amino acid sequence of NB467-7) is:
QVQLVESGGGSVHPGGSLRLSCAGSGFTLAGYAIGWFRQAPGKEREGVSCINSSG
GSTYYADSVKGRFTISRDNAKNTVYLQMNSLKPEDTAVYYCAADVYYSGSYLYRSC
NPTESKYWGQGTQVTVSS;
SEQ ID No: 3 (the amino acid sequence of NB467-71) is:
EVQVVESGGGLVQSGGSLRLSCVASGFNLDHYAIGWFRQIPGKEREGVSCISSGG
DSTFYIDSVKGRFTISRDNAKNTVYLQMNSLKPEDTAVYHCAADVYYSGNYLYRSCN
PHESKYWGQGTQVTVSS;
SEQ ID No: 4 (the amino acid sequence of NB467-88) is:
EVQVVESGGGLVQPGGSLRLSCAASGFRLNYYAIGWFRQAPGKEREGVSCISSG
GDRIYYADSVKGRFTISRDNAKNTVYLQMNSLKPEDTAVYYCAADLYYSGSYYYRS
CNPAESGYWGQGTQVTVSS;
SEQ ID No: 18 (the nucleotide sequence of NB467-7) is:
CAGGTGCAGCTGGTTGAATCTGGCGGAGGATCTGTGCACCCTGGCGGATCTCT
GAGACTGTCTTGTGCCGGCAGCGGCTTTACACTGGCCGGATATGCCATCGGCTGGT
TCAGACAGGCCCCTGGCAAAGAGAGAGAGGGCGTCAGCTGCATCAATAGCTCTG
GCGGCAGCACCTACTACGCCGACTCTGTGAAGGGCAGATTCACCATCAGCCGGGA
CAACGCCAAGAACACCGTGTACCTGCAGATGAACAGCCTGAAGCCTGAGGACAC
CGCCGTGTACTATTGTGCCGCCGACGTGTACTACAGCGGCAGCTACCTGTACAGAA
GCTGCAACCCCACCGAGAGCAAGTATTGGGGCCAGGGCACACAAGTGACCGTGT
CTAGT;
SEQ ID No: 19 (the nucleotide sequence of NB467-71) is:
GAGGTGCAGGTTGTGGAATCTGGCGGAGGACTGGTTCAGTCTGGCGGCTCTC
TGAGACTGAGCTGTGTGGCCAGCGGCTTCAACCTGGATCACTATGCCATCGGCTG
GTTCAGACAGATCCCCGGCAAAGAGAGAGAGGGCGTCAGCTGTATCAGCAGCGG
CGGAGATAGCACCTTCTACATCGACAGCGTGAAGGGCAGATTCACCATCAGCCGG
GACAACGCCAAGAACACCGTGTACCTGCAGATGAACAGCCTGAAGCCTGAGGAC
ACCGCCGTGTATCATTGTGCCGCCGACGTGTACTACAGCGGCAACTACCTGTACAG
AAGCTGCAACCCTCACGAGAGCAAGTACTGGGGCCAGGGCACACAAGTGACCGT
GTCATCT;
SEQ ID No: 20 (the nucleotide sequence of NB467-88) is:
GAGGTGCAGGTTGTGGAATCTGGCGGAGGACTGGTTCAGCCTGGCGGATCTC
TGAGACTGTCTTGTGCCGCCAGCGGCTTCCGGCTGAATTACTATGCCATCGGCTGG
TTCAGACAGGCCCCTGGCAAAGAGAGAGAGGGCGTCAGCTGTATCAGCTCTGGC
GGCGACAGAATCTACTACGCCGACAGCGTGAAGGGCAGATTCACCATCAGCCGGG
ACAACGCCAAGAACACCGTGTACCTGCAGATGAACAGCCTGAAGCCTGAGGACA
CCGCCGTGTATTACTGTGCCGCCGACCTGTACTACAGCGGCAGCTACTACTACAGA
AGCTGCAACCCTGCCGAGAGCGGCTATTGGGGACAGGGAACACAAGTGACCGTG
TCCTCT.

### Example 2: Binding of Anti-BAFFR Nanobody to 293T Cells Overexpressing BAFFR

The binding affinity of the 3 anti-BAFFR nanobodies NB467-7, NB467-71 and NB467-88 obtained in Example 1 were characterized by flow cytometry.

Specifically, HEK-293T-BAFFR cells were collected from the cell culture flask, washed twice with a FACS buffer and centrifuged. The cells were resuspended in a suitable volume of staining solution for flow cytometry, so that the final cell concentration per tube reached 2×10⁵ cells/mL.

The antibodies NB467-7, NB467-71, and NB467-88 produced in Example 1 and the control antibody were diluted to 200 nM, respectively. 50 μL of the antibody solution was added into a test tube with HEK-293T-BAFFR cells, mixed uniformly, and incubated at 4° C. for 60 min. After incubation, the cells were washed once with a FACS buffer.

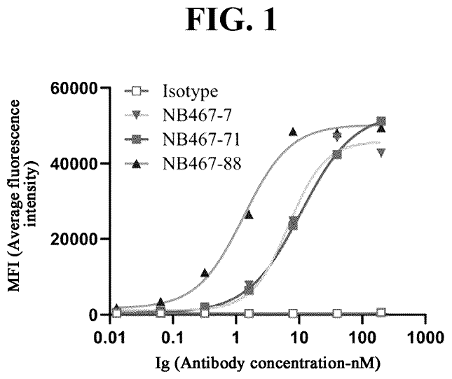

The anti-human FC-647 secondary antibody (Jackson, 109-605-003) was diluted according to the instructions, and 100 μL of the diluted secondary antibody was added to a sample tube. After uniform mixing, the system was incubated at 4° C. for 60 min. After incubation, the cells were washed 3 times with FACS, and the cell precipitate obtained by centrifugation was resuspended in 200 μL of PBS and detected by flow cytometry (Sony, SA3800). The detection results are shown in Table 3 and FIG. 1.

TABLE 3
Binding of nanobody to 293T cells overexpressing BAFF (EC₅₀)

| | NB467-7 | NB467-71 | NB467-88 |
|---|---|---|---|
| EC₅₀ (nM) | 6.727 | 10.61 | 1.315 |

FIG. 1 shows the average fluorescence intensity (MFI) value of each antibody. It is confirmed that compared with the control antibody, the 3 nanobodies of the present disclosure have stronger binding ability to 293T cells that overexpress human BAFFR. Table 3 shows the EC₅₀ value of the nanobody of the present disclosure.

### Example 3: Construction of BAFFR-CAR-T Cells

### I. Construction of CAR Lentiviral Expression Vector

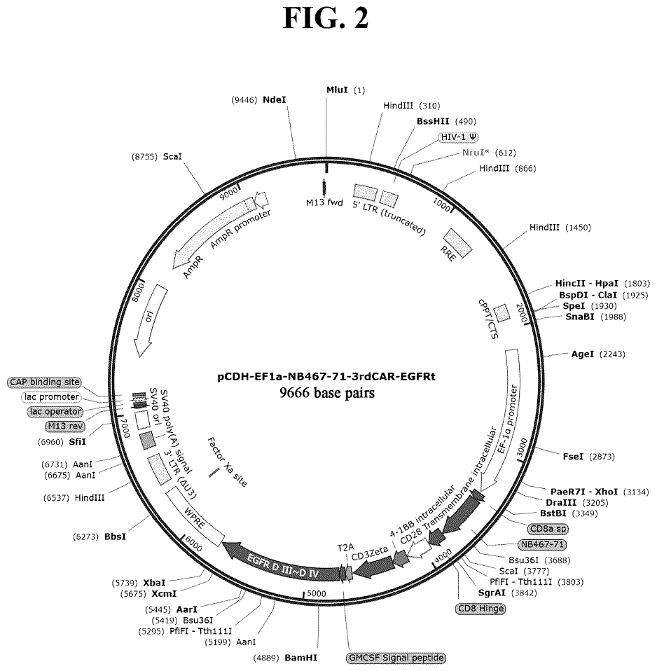

After codon optimization, the obtained BAFFR antibody sequence and related gene fragments of the hinge region, the transmembrane region and the intracellular signal region were constructed into a lentiviral expression vector for the chimeric antigen receptor. The constructed vector PCDH lentiviral expression vector (pCDH-EF1a) was selected, as shown in FIG. 2. Sanger sequencing confirmed that the inserted sequence was correct, then lentivirus packaging was carried out.

### II. CAR Lentivirus Packaging

1) A 15 cm cell culture dish was prepared. 1×10⁷ 293T cells were inoculated, and a complete culture medium (high-glucose DMEM with 10% FBS) was added. The system was incubated overnight in an incubator at 37° C. and 5% CO₂.
2) LVTransm, lentiviral expression plasmid (CAR plasmid) and lentiviral packaging plasmid mix were removed from the freezer, thawed at room temperature, and beaten up and down with a pipette to mix them completely. The PBS buffer was taken out and warmed to room temperature. 2 mL PBS was transferred to one well of a 6-well plate, and 20 μg of lentiviral expression plasmid and 30 μL of lentiviral packaging plasmid mix were added respectively. After mixing uniformly by beating up and down with a pipette, 150 μL LVTransm was added, immediately beaten up and down with a pipette to mix them uniformly, and let stand at room temperature for 10 min.
3) The DNA/LVTransm complex was added dropwise to a 15 cm culture dish, and the culture dish was gently shaken to fully mix them. The culture dish was allowed to stand in an incubator at 37° C. and 5% CO₂. After 6-8 h of culture, the culture medium containing the transfection reagent was removed, and replaced by a fresh complete culture medium.
4) After 48 h of continuous culture, the culture supernatant containing viruses in the culture dish was collected, filtered through a 0.45 μm filter membrane, transferred to a centrifuge tube, balanced, and centrifuged at 40000×g and 4° C. for 1.5 h. After centrifugation, the liquid in the centrifuge tube was carefully sucked off in a biosafety cabinet, 1 mL of a PBS buffer was added to resuspend the precipitate, and the viruses were stored at −80° C.

### III. Separation of Peripheral Blood T Cells

1) The anticoagulated blood sample was transferred into a 50 mL sterile centrifuge tube, and centrifuged at 800×g for 20 min after the cap was screwed tightly.
2) After centrifugation, the centrifuge tube was taken out, and the upper pale yellow serum layer was removed while the centrifuge tube was avoided to be shaken violently or turned upside down. PBS of the same volume was added to the lower peripheral blood cell layer and mixed evenly by gently turning it upside down.
3) The lymphocyte separation buffer was taken and turned upside down several times to fully mix them. 20 mL of the lymphocyte separation buffer was added into a 50 mL centrifuge tube, and then an equal volume of the blood sample diluted in Step 2 was carefully slowly added to the upper layer of the lymphocyte separation buffer along the tube wall by using a pipette, to avoid the mixing of the separation reagent and the blood sample. The system was then centrifuged at 800×g for 20 min.
4) After centrifugation, the centrifuge tube was gently taken out, the middle white monocyte layer was pipetted into a new sterile centrifuge tube, and an equal volume of physiological saline was added and gently mixed uniformly and centrifuged at 800×g for 5 min. After centrifugation, the supernatant was removed, the PBMCs were washed once, and the cell density was adjusted to 5×10⁷ cells/mL. The cells were transferred to a 2 mL cell cryopreservation tube, and cryopreserved in an amount of 1 mL/tube.

### IV. Production of CAR-T Cells

1) CD3/CD28 Dynabeads was washed twice with PBS.
2) A proper amount of Dynabeads were added to PBMC, gently mixed and incubated at room temperature for 20 min.
3) A 2 mL cell cryopreservation tube was inserted into a magnetic pole, and let to stand at room temperature for 1 min. While the cryopreservation tube was inserted into the hole of the magnetic pole, it was gently inverted, and the liquid in the tube was poured out.
4) The cell cryopreservation tube was removed from the magnetic pole, a proper amount of X-Vivo 15 medium (containing 200 IU/mL IL2, 10 ng/ml IL7, and 5 ng/ml IL15) was added. The mixture of cells and beads was resuspended by using a pipette, and the cell density was adjusted to 0.5-1×10⁶ cells/mL. The cells were transfer to a 6-well plate.
5) After the cells were placed in an incubator at 37° C. and 5% CO₂ for 48 h, the cell density was adjusted to 1×10⁶ cells/mL.
6) The lentivirus was removed from a freezer at an ultra-low temperature of −80° C. and thawed quickly.
7) Polybrene was added to 2 mL of the prepared T cells (1×10⁶ cells/mL), to give a final concentration of 6 μg/mL. 300 μL lentivirus was added, and mixed well by gently beating with a pipette. The culture dish was sealed with a sealing membrane, and centrifuge at 800×g for 1 h at room temperature.
8) After centrifugation, the cells were further cultured for 24 h, and the culture medium of the T cells was changed.
9) After 24 h of further culture, the EGFR antibody was incubated and the CAR-T cell positive rate was detected by flow cytometry.
10) The remaining CAR-T cells were further incubated, and the beads/cell clusters in the system were gently beaten every day until they were completely separated. When the cell density was greater than 1×10⁶ cells/mL, X-Vivo 15 medium (containing 200 IU/mL IL-2, 10 ng/ml IL-7, and 5 ng/ml IL-15) was added, and the cell density was adjusted to 0.5-0.7×10⁶ cells/mL.

### Example 4: Detection of CAR-T Cells

### I. Detection of CAR Positive Rate

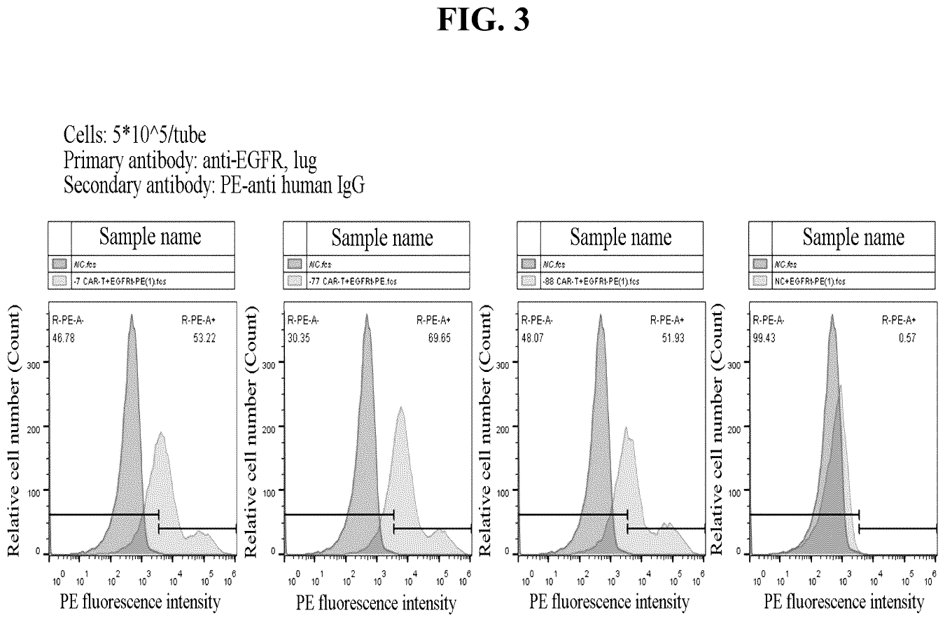

1) After centrifugation at 500×g for 5 min, the cell precipitate (2 tubes of cells were prepared, with 5×10⁵ cells/tube) was collected.
2) The cell precipitate was washed 3 times with a PBS buffer containing 0.5% BSA, and centrifuged at 500×g for 5 min each time.
3) The cell precipitate was resuspended with 100 μL of diluted EGFR antibody (1 μg/tube) and incubated at room temperature for 60 min. After centrifugation at 500×g for 5 min, the supernatant (one tube of cells was used as the control, and only PBS containing 0.5% BSA was added in this step).
4) The cell precipitate was washed 3 times with a PBS buffer containing 0.5% BSA, and centrifuged at 500×g for 5 min each time.
5) The cell precipitate (all cells were incubated with the secondary antibody) was resuspended with 100 μL of diluted PE anti human IgG (1:500 dilution), and incubated for 45 min in the dark. After centrifugation at 500×g for 5 min, the cell precipitate was collected.
6) The cell precipitate was washed 3 times with a PBS buffer containing 0.5% BSA, and centrifuged at 500×g for 5 min each time.
7) Finally, the cell precipitate was resuspended with 400 μL of PBS, and analyzed by flow cytometry. The specific results are shown in FIG. 3.

The results in FIG. 3 shows that after detection using EGFR antibody, the CAR positive rate is 30%. The in-vitro killing with CAR-T can be carried out. The NB467-7 CAR-T positive rate is 53.22%, the NB467-77 CAR-T positive rate is 69.65%, and the NB467-88 CAR-T positive rate is 51.93%.

### II. Lysis of Target Cells by CAR-T Cells

1) The target cells Nalm-6-Luc were resuspended in a complete medium (RPMI 1640+10% FBS) and the cell density was adjusted to 2×10⁵ cells/mL. The target cells were inoculated into a new 96-well plate in an amount of 100 μL/well. 100 μL of sterile water was added to wells of a 96-well plate that have not been used for four weeks, to prevent the water in the middle experimental well from evaporation. The plate was incubated overnight in an incubator with 5% CO₂ at 37° C.
2) The produced CAR-T cells were collected by centrifugation and resuspended in 1640 culture medium with 10% FBS. The 96-well plate was removed from the incubator, the culture medium in the well was completely aspirated off, and the cells were gently washed with sterile PBS. Then CAR-T cells were added according to different E/T ratios (1:1, 2.5:1, 5:1, or 10:1), and the final volume was made up to 200 μL/well. Maxi lysis was inoculated with the same number of target cells but without CAR-T cells. The plate was incubated in an incubator with 5% CO₂ at 37° C. for 18 h.
3) After culture, the plate was taken out of the incubator, and the supernatant was collected by centrifugation and stored in a freezer at −80° C. for detection of the expression of IL-2 and IFN-γ by Elisa. Bright-Glo™ was added to the cells, and the luciferase activity was detected to reflect the lytic ability of recombinant CAR-T cells to the target cells.
4) Calculation formula of target cell lysis percentage:

$$Lysis \% = ( 1 - \frac{R L {U}_{Sample}}{R L {U}_{M ax}} ) \times 100 \% .$$

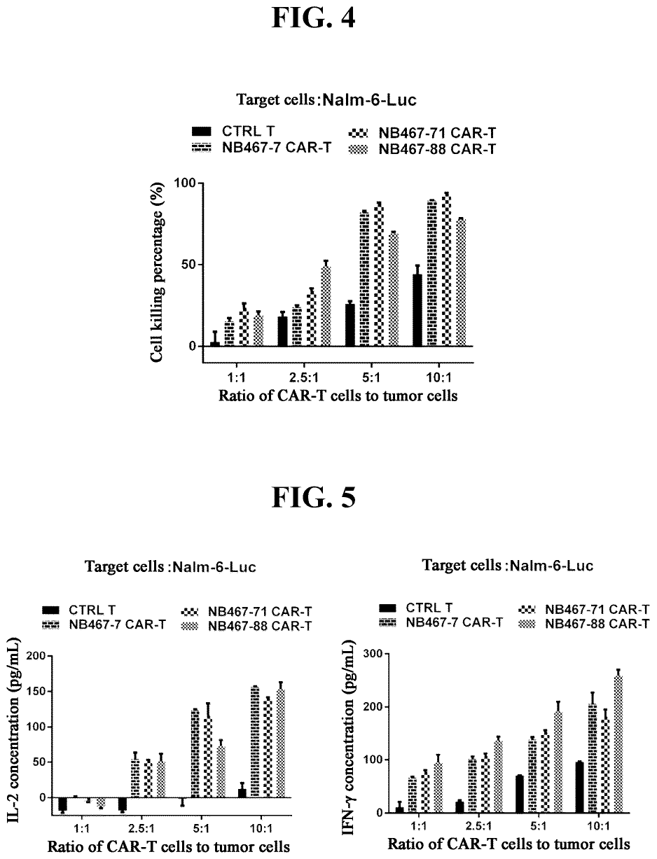

5) The specific results are shown in FIG. 4.

The results in FIG. 4 shows that when the target cells Nalm-6-Luc are co-incubated with CAR-T cells at an effector-to-target ratio E/T of 1:1, 2.5:1, 5:1, or 10:1, NB467-7 CAR-T, NB467-71 CAR-T and NB467-88 CAR-T cells have a powerful killing effect on Nalm-6-Luc compared with the blank T cells.

### III. Detection of CAR-T Cytokine Secretion Level

100 μL of the supernatant from co-incubation was used for the detection of IL-2 and IFN-γ respectively. According to the operation instructions of the ELISA detection kit for IL-2 and IFN-γ, the secretion of IL-2 and IFN-γ was detected. The specific results are shown in FIG. 5.

The results in FIG. 5 shows that compared with the blank T, the secretion of IL-2 and IFN-γ by NB467-7 CAR-T, NB467-71 CAR-T and NB467-88 CAR-T cells is significantly enhanced after being stimulated by Nalm-6-Luc cells.

The present disclosure provides a CAR-T cell targeting BAFFR, which mainly targets autoreactive B cells, and provides a new option for the treatment of autoimmune diseases and some hematological tumors.

In the description of the specification, the description with reference to the terms “an embodiment”, “some examples” and so on means that a specific feature, structure, materials or characteristics described in connection with the embodiment or example are embraced in at least one embodiment or example of the present disclosure. In the specification, exemplary descriptions of the foregoing terms do not necessarily refer to the same embodiment or example. In addition, the described specific feature, structure, materials, or characteristics may be combined in a proper manner in any one or more of the embodiments or examples.

Preferred embodiments of the present disclosure have been described above; however, the present disclosure is not limited thereto. Various variations and changes can be made by those skilled in the art to the present disclosure. Any changes, equivalent replacements and improvements made without departing from the spirit and principle of the present disclosure. Any modification, equivalent substitution, and improvement made without departing from the spirit and principle of the present disclosure are embraced in the scope of protection of the present disclosure.

What is claimed is:

1. A chimeric antigen receptor targeting BAFFR, comprising, from the N end to the C end, a signal peptide region, an antigen binding domain targeting BAFFR, a hinge region, a transmembrane domain, a costimulatory domain and a signal transduction domain connected in sequence, wherein the costimulatory domain comprises a CD28 costimulatory domain and a 4-1BB costimulatory domain which are connected, and the signal transduction domain is ITAM1 of CD3ζ.

2. The chimeric antigen receptor targeting BAFFR according to claim 1, wherein the signal peptide region has an amino acid sequence as shown in SEQ ID No: 1.

3. The chimeric antigen receptor targeting BAFFR according to claim 1, wherein the antigen binding domain targeting BAFFR is a nanobody targeting BAFFR, and the nanobody targeting BAFFR has an amino acid sequence as shown in SEQ ID No: 2, SEQ ID No: 3, or SEQ ID No: 4.

4. The chimeric antigen receptor targeting BAFFR according to claim 1, wherein the hinge region is a CD8 hinge domain having an amino acid sequence as shown in SEQ ID No: 5; and/or
the transmembrane domain is a CD28 transmembrane domain having an amino acid sequence as shown in SEQ ID No: 6; and/or
the CD28 costimulatory domain has an amino acid sequence as shown in SEQ ID No: 7; and/or
the 4-1BB costimulatory domain has an amino acid sequence as shown in SEQ ID No: 8; and/or
the ITAM1 of CD3ζ has an amino acid sequence as shown in SEQ ID No: 9.

5. An isolated nucleic acid, comprising a nucleotide sequence for expressing the chimeric antigen receptor targeting BAFFR according to claim 1.

6. The isolated nucleic acid according to claim 5, comprising a nucleic acid fragment having a nucleotide sequence as shown in SEQ ID NO: 10.

7. A recombinant vector, comprising the isolated nucleic acid according to claim 5.

8. A CAR-T cell, comprising the isolated nucleic acid according to claim 5.

9. Use of a chimeric antigen receptor targeting BAFFR in drugs, comprising use of the chimeric antigen receptor targeting BAFFR according to claim 1 in preparing drugs for treating immune-related diseases.

10. A pharmaceutical composition, comprising an expression vector for expressing the chimeric antigen receptor targeting BAFFR according to claim 1.
